(19) United States
(12) Patent Application Publication
(10) Pub. No.: US 2026/0021288 A1
(43) Pub. Date: Jan. 22, 2026

(54) Minimally Invasive Heart Pump for Assisting Systolic and Diastolic Pump Function with Modular Adjustable Strain Construct Insertion

(71) Applicant: Lifebridge Technologies LLC, Dayton, OH (US)

(72) Inventor: Mark P. Anstadt, Kettering, OH (US)

(21) Appl. No.: 19/339,227

(22) Filed: Sep. 24, 2025

Related U.S. Application Data
(63) Continuation-in-part of application No. 18/530,157, filed on Dec. 5, 2023, now Pat. No. 12,440,338.
Application No. 18/530,157 is a continuation-in-part of application No. 18/447,786, filed on Aug. 10, 2023, now Pat. No. 12,115,363.
Application No. 18/447,786 is a continuation-in-part of application No. 18/509,260, filed on Nov. 14, 2023.
Application No. 18/509,260 is a continuation-in-part of application No. 17/825,343, filed on May 26, 2022.
Application No. 17/825,343 is a continuation-in-part of application No. 17/208,776, filed on Mar. 22, 2021, now Pat. No. 11,383,076.
Application No. 18/530,157 is a continuation-in-part of application No. 18/160,963, filed on Jan. 27, 2023, now Pat. No. 11,896,812.
(60) Provisional application No. 63/086,478, filed on Oct. 1, 2020.

(51) Int. Cl. A61M 60/117; A61M 60/289; A61M 60/465; A61M 60/839

(52) U.S. Cl. CPC A61M 60/117; A61M 60/289; A61M 60/465; A61M 60/839

(57) ABSTRACT

A system and method for the installation and operation of a cardiac assist device. Flexible guides are advanced into a prepared space using minimally invasive techniques. A cardiac assist device is advanced into position in the pericardial area along the flexible guides. Once in position, the cardiac assist device is activated while still engaged with the flexible guides. The flexible guides provide structural integrity to the cardiac assist device needed in order for the cardiac assist device to function properly. The forces supplied to the heart by the cardiac assist device are affected by the presence of the flexible guides. The structure of the flexible guides, the position of the flexible guides, and the structure of the cardiac assist device are customized to supply the forces needed by a particular heart in order to assist the heart in pumping more efficiently.

### RELATED APPLICATIONS

This application is a continuation-in-part of U.S. patent application Ser. No. 18/530,157, filed Dec. 5, 2023, which is a continuation-in-part of U.S. patent application Ser. No. 18/447,786 filed Aug. 10, 2023.

This application is also a continuation-in-part of U.S. patent application Ser. No. 18/509,260 filed Nov. 14, 2023, which is a continuation-in-part of co-pending U.S. patent application Ser. No. 17/825,343 filed May 26, 2022, which is a continuation-in-part of U.S. patent application Ser. No. 17/208,776 filed Mar. 22, 2021, now U.S. Pat. No. 11,383,076, which claimed the benefit of U.S. Provisional Patent Application No. 63/086,478 filed Oct. 1, 2020.

This application is a further continuation-in-part of U.S. patent application Ser. No. 18/160,963, filed Jan. 27, 2023.

### BACKGROUND OF THE INVENTION

### 1. Field of the Invention

In general, the present invention relates to cardiac assist systems and methods that help the heart pump blood by applying forces to the exterior of the heart. More particularly, the present invention relates to the structure of the cardiac assist systems, their methods of operation, and their methods of insertion into the body.

### 2. Prior Art Description

Proper heart function requires the ventricular chambers to fill, termed diastolic function, and empty, termed systolic function. There are many instances when a heart needs assistance to properly maintain blood flow in a patient. Often hearts that are diseased, failing, or have stopped need the application of a cardiac assist system to prevent a patient from dying. Furthermore, utilizing a proper cardiac assist device can assist in systolic heart function, diastolic heart function, and/or the recovery of such deficiency of failing hearts. To minimize the dangers of thromboembolic events, hemolysis, immune reactions, infections, and the need for anticoagulation it is preferable that the pumping of the heart be assisted by applying forces to the external surfaces of the heart with no direct contact to the blood being pumped. However, in order to apply forces directly to the exterior of the heart, a cardiac assist device must be surgically introduced into the pericardial area.

In simple terms, the term “pericardial area” implies the area just outside the heart's epicardium or outer surface. Within the pericardial area, the heart is normally surrounded by the pericardium sac. The pericardial sac typically contains pericardial fluid, which protects and lubricates the heart. The pericardial area can contain fibrous connective tissue or scar tissue that extends between the epicardium of the heart and the pericardial sac. If the heart has been diseased or has been previously operated upon, there is often scar tissue within the pericardial area. Therefore, there may exist situations where only part of the heart's surface is amenable to the insertion or placement of a non-blood contacting heart pump. The most common example is that of a prior sternotomy or where a critical internal mammary artery bypass graft is in place. In either of these circumstances, the placement of a non-blood contacting heart pump that only lies over part of the ventricular surface would be on the back side or posterior aspect of the heart. Here the heart pump could provide reasonable support without risking the area of the internal mammary graft. Additionally, the posterior action of the heart pump would be countered by the natural location of the sternum that would facilitate the function of the heart pump. Such a modular approach would work in any selected region around the heart as well.

Accordingly, it can be difficult to position a cardiac assist device into the pericardial area. Cardiac assist devices, such as heart pump devices, are generally designed to contact, or fit around, the ventricles of a surgically exposed heart. Accordingly, such constructs are typically applied using open heart surgical techniques. Likewise, creating the required opening within the pericardial area is also often accomplished using open surgical techniques. However, open heart surgeries have many inherent problems. Open heart surgeries are highly invasive and can result in significant blood loss and infection risk. Furthermore, open heart surgeries require longer surgical times, longer stays at the hospital, and longer recovery periods. Lastly, open heart surgeries often leave visible scars on the chest of the patient.

It is for these and other reasons that many physicians and patients prefer minimally invasive surgical procedures. Minimally invasive heart surgeries involve making small incisions in the chest to reach the heart. The obvious problem is that heart pumps and like constructs that directly apply forces to the heart can be too large to use in traditional minimally invasive procedures. The result is that the surgical opening must be enlarged to accommodate the construct being inserted. Consequently, the minimally invasive procedure becomes more invasive than desired. This problem can be addressed by a surgical team in two ways. First, a smaller or partial construct can be used that is small enough to pass through the minimally invasive incision. Additionally, a collapsible heart pump can be used. In both scenarios, some operational aspects of the heart pump must be constructed in order to make the heart pump collapsible. Furthermore, the versatility of the heart pump is important to avoid injury to areas where insertion is deemed to result in potential injury to a critical graft or scarring is too severe. Finally, a means for imaging the position and functionality is needed to assess verify proper position and functionality is needed.

The heart is a complex organ that both empties and fills as it contracts and relaxes. In order for a heart to pump blood effectively and efficiently during the cardiac cycle, an unhealthy heart often needs assistance during both systole and diastole. In the prior art, most cardiac assist devices only help a heart contract during systole. The devices provide no assistance in the diastole when the heart is trying to refill with blood. However, if the heart does not properly refill with blood in diastole, then there is less blood to pump in systole. The result is that the overall efficiency of the heart decreases, regardless to the forces applied during systole. Accordingly, even if the most effective cardiac assist device is used during systole, it would do little good if the heart is incapable of refilling during diastole.

Computer imaging software has allowed intricate three dimensional understanding of conformational changes in the heart which can be characterized in three dimensional strain analyses. The interactions of a cardiac assist device and the heart can also be analyzed by software to calculate the ideal compression and expansion forces that the cardiac assist device should apply to the heart during both systole and diastole. The forces provided by the cardiac assist device can be further refined by controlling the pneumatic forces that operate the cardiac assist device to adjust for physiological changes imposed by the cardiovascular system.

In order for a cardiac assist device to assist a heart in emptying during systole, the cardiac assist device must generally apply compressive forces to the exterior of the heart. There are several cardiac assist devices proposed for commercial use that are designed to apply compression forces to the heart and are also designed for use in minimally invasive surgical procedures. However, these prior art devices are collapsible structures that lack the physical integrity to apply anything but limited compressive forces to the heart. Furthermore, such prior art devices do not allow for inserting a pump that can selectively act on only part of the heart's surface when complete circumferential insertion is deemed not prudent. Such prior art devices are exemplified by U.S. Pat. No. 10,463,496 to Criscone and U.S. Pat. No. 11,511,102 to Criscone.

In order for a cardiac assist device to assist a heart in refilling during diastole, the cardiac assist device must be able to assist the heart in expanding. In order for a cardiac assist device to assist the heart in expanding during diastole, the cardiac assist device must have the structural integrity needed to apply expanding forces to the heart without collapsing. Normally, this requires that the cardiac assist device have a ridged or semi-rigid shell such as that disclosed in U.S. Pat. No. 3,455,298, to Dr. George Anstadt. In the medical community, the device described in U.S. Pat. No. 3,455,298 is known as the Anstadt cup. The Anstadt cup is a cup-shaped construct that fits over the ventricles of the heart. The Anstadt cup has a stiff outer shell and an inflatable inner membrane. The outer shell and the inflatable membrane are placed around the ventricles of the heart. When the inflatable membrane expands, the inflatable membrane compresses the heart, therein helping the heart to empty. When the membrane deflates, there is a negative pressure that is created between the tissue of the heart and the stiff outer shell. The outer shell has the integrity to resist the resulting forces. As such, the forces are transferred to the heart, wherein the forces assist the heart in filling during diastole.

Since the Anstadt cup has a rigid outer shell that is sized to fit over the ventricles of the heart, the Anstadt cup cannot be collapsed and therefore utilized in a minimally invasive procedure. Furthermore, since the outer shell of the Anstadt cup is rigid, the assistance provided to the heart's pump function is less than optimal. When the heart has an inherent pump function, the heart does more than fill and empty. During the cardiac cycle, different areas of the heart elongate, twist, and contract. A cardiac assist device that uses a rigid shell has a very limited ability to follow the complex movements of the heart's surfaces as those surfaces elongate, twist and/or contract. This is important in conditions where the heart has no pump function and is changing its conformation in response to the device's forces, as well as when the heart is exhibiting inherent pump function, and the device is aiding in promoting physiologic diastolic and systolic pump function. The result is an application of forces that are a compromise between what is mechanically achievable and what is needed to properly follow the heart's natural strain dynamics during the cardiac cycle.

A large need therefore exists for a cardiac assist device that can be utilized to apply the proper forces to a heart in order to optimize the heart's ability to both fill and empty, wherein the cardiac assist device is also collapsible so as to be usable during a minimally invasive procedure. A need further exists for a cardiac assist device that can be inserted on, or near, a heart where the pericardium area is complicated with fibrous tissue and/or scar tissue. A further need also exists for the device to be able to completely encompass the heart or partially encompass the heart depending on factors such as pre-existing scarring or critical implants from prior treatment or surgery. The device needs to have the ability to aid the heart in both heart filling and emptying to be efficacious. Finally, a small imaging means such as a video camera or ultrasound probe needs to be able to assess the device position around the heart and its functionality. These needs are met by the present invention as described and claimed below.

### SUMMARY OF THE INVENTION

The present invention is a cardiac assist device and its associated methods of installation and operation. A space is prepared in the pericardial area which ideally provides access around the entire ventricular surface or only a portion of the surface depending on the circumstances. Flexible guides are advanced into the prepared space using minimally invasive techniques. A small video camera or ultrasound probe can be used to assess position and functionality. A cardiac assist device is provided that can be collapsed and introduced in vivo through the same incision as is being used for the flexible guides. The cardiac assist device is advanced into position in the pericardial area along the flexible guides. The flexible guides move the cardiac assist device into operable position where the cardiac assist device can act upon the heart. The flexible guides can be used to facilitate or guide entry of a small video camera or ultrasound to evaluate positioning or function of the device. The guides can also be used to connect modular components when a fully encompassing pump cannot be safely inserted.

Once in position, the cardiac assist device is activated while still engaged with the flexible guides. An ultrasound probe or camera can be inserted at any time to assess position and functionality. The flexible guides can be exchanged as needed to provide optimal structural integrity to the cardiac assist device needed in order for the cardiac assist device to function properly. The dynamic strain forces applied to the heart by the cardiac assist device are affected by the presence of the flexible guides. The structure of the flexible guides, the position of the flexible guides and the structure of the cardiac assist device are engineered to provide forces that meet the strain force requirements of a particular heart in order to assist the heart in pumping more efficiently.

The cardiac assist device contains flexible membranes within an outer shell. The flexible guides within the cardiac assist device act like springs and promote the outer shell of the cardiac assist device to remain expanded toward an ideal end-diastolic diameter. This ideal end-diastolic diameter can be defined within a range of a first maximum diameter and a second minimum diameter. The outer shell also provides a cushion during inflation of the inflatable member during systolic compression to facilitate conformation to the heart's surface during systolic actuation. The forces of the pneumatic drive, the inflatable membranes, the outer shell, and the flexible guides all combine to achieve the dynamic strain force requirements for assisting the heart's pump function. This can be further guided by evaluating the strain dynamics of the heart while it is being assisted, which can be performed by inserting a small video camera or ultrasound probe into the guides or into the tubing of the drive system.

### BRIEF DESCRIPTION OF THE DRAWINGS

For a better understanding of the present invention, reference is made to the following description of exemplary embodiments thereof, considered in conjunction with the accompanying drawings, in which:

FIG. 1 shows a first exemplary embodiment of a cardiac assist device shown in conjunction with a heart;

FIG. 2 shows a top view of the exemplary cardiac assist device to illustrate guide positions and range of expansion/contraction;

FIG. 3 is a partial cross-sectional view of the exemplary cardiac assist device to illustrate passage of flexible guides through conduits in the cup structure;

FIG. 4 shows a top view of the exemplary cardiac assist device helping the heart empty;

FIG. 5 shows a top view of the exemplary cardiac assist device helping the heart fill;

FIG. 6 illustrates the methodology of positioning the flexible guides near the heart;

FIG. 7 illustrates a cup assembly in a collapsed condition being advanced along the flexible guides;

FIG. 8 illustrates a cup assembly in an open configuration being reinforced by the flexible guides;

FIG. 9 shows a second exemplary embodiment of a cardiac assist device;

FIG. 10 shows a third exemplary embodiment of a cardiac assist device; and

FIG. 11 shows a fourth exemplary embodiment of a cardiac assist device with a modular construction.

### DETAILED DESCRIPTION OF THE DRAWINGS

Although the present invention system and method can be varied in different ways, only a few embodiments are illustrated. The exemplary embodiments are being shown for the purposes of explanation and description. The exemplary embodiments are selected in order to set forth some of the best modes contemplated for the invention. The illustrated embodiments, however, are merely exemplary and should not be considered limitations when interpreting the scope of the appended claims.

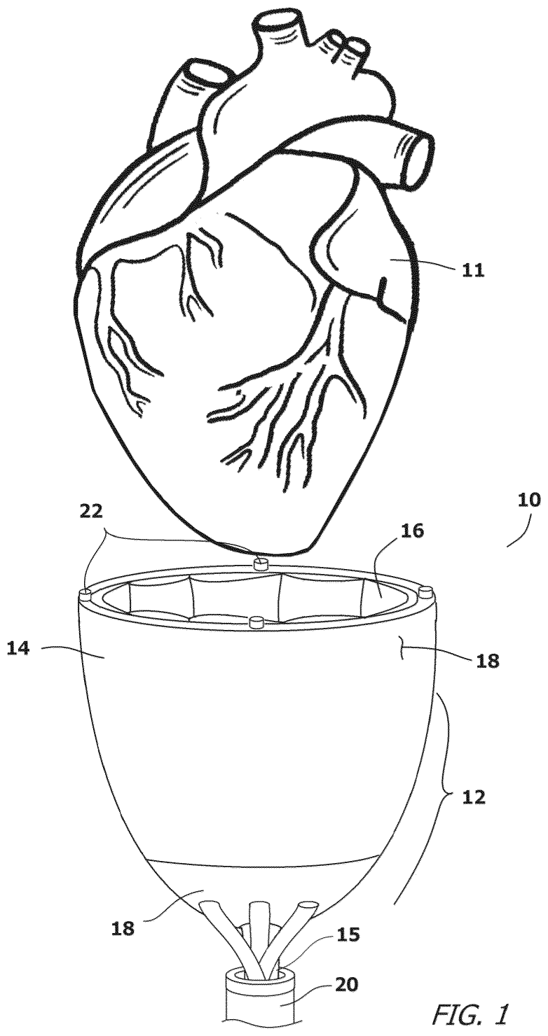

Referring to FIG. 1, an improved cardiac assist device 10 is shown in conjunction with a heart 11. The cardiac assist device 10 contains a cup assembly 12. The cup assembly 12 includes an outer shell 14 that is internally lined with one or more inflatable membranes 16. The outer shell 14 is set upon a base 18 to complete the cup assembly 12. The outer shell 14, the inflatable membranes 16, and the base 18 are all capable of being collapsed, or folded, and passed through a surgical insertion tube 20 using minimally invasive surgical equipment and techniques.

One or more tubes 15 extend from the cup assembly 12. The tubes 15 are used to supply pneumatic or hydraulic pressure to the inflatable membranes 16. The tubes 15 are also used to supply suction to the cup assembly 12. The suction is used to drain blood and fluids from the cup assembly 12. Furthermore, the suction is primarily used to retain the cup assembly 12 in place over the heart 11 as the cardiac assist device 10 is activated. The suction also helps guide the cup assembly 12 over the heart 11 as the cardiac assist device 10 is advanced into the body.

The outer shell 14 of the cup assembly 12 is reinforced with a plurality of flexible guides 22. As will be explained, the flexible guides 22 are used to position the cup assembly 12 around the ventricles of the heart 11. Once used to guide the cup assembly 12 into place, the flexible guides 22 can be withdrawn or left in place. Also, they can serve as guides for a small video camera or ultrasound probe to assess position and functionality. When the flexible guides 22 are left in place they can provide structural integrity and reinforce the outer shell 14 of the cup assembly 12. Each of the flexible guides 22 has an inherent spring constant that enables the flexible guides 22 to be elastically displaced without permanent bending. It will therefore be understood that the spring constant inherent in the flexible guides, resists the expansion of the shell 14 and the contraction of the shell 14 without preventing either expansion or contraction of the shell 14 within a range.

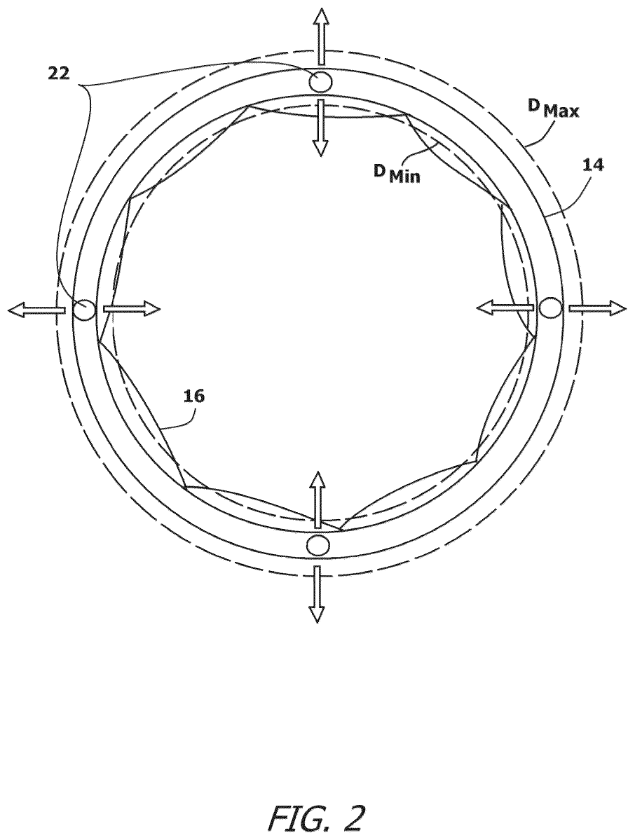

Referring to FIG. 2 in conjunction with FIG. 1, it can be seen that the presence of the flexible guides 22 in the outer shell 14 enables the outer shell 14 to elastically expand and contract within a range between a maximum diameter Dmax and a minimum diameter Dmin. However, the presence of the flexible guides 22 prevents the outer shell 14 from over expanding beyond the maximum diameter Dmax or collapsing under the minimum diameter Dmin. The spring constant inherent in the flexible guides 22 also affects the strain characteristics embodied by the outer shell 14 and the inflatable membranes 16 when all components are considered as an integrated unit. Accordingly, the presence of the flexible guides 22 effect the three dimensional strain forces that both the outer shell 14 and the inflatable membranes 16 apply to the heart 11.

During systole, the inflatable membranes 16 are inflated by various pressures. This causes the inflatable membranes 16 to expand and conform to the heart surface 11. The inflatable membranes 16 also press against the outer shell 14 and act to expand the outer shell 14. The outer shell 14 resists the expansion as a function of the elasticity of the outer shell's material and the spring constant of the flexible guides 22. Accordingly, the three dimensional stain forces applied to the heart are a combination of the drive system pressure profile delivery and both the resulting forces directly applied by the expanding inflatable membranes 16 and the reciprocal resistance forces of the outer shell 14 and the flexible guides 22.

During diastole, the inflatable membranes 16 are deflated. This causes the inflatable membranes 16 to contract and pull the heart 11 surface and augment filling. This is explained by cohesive forces facilitated by the vacuum delivered thru the apical port. The adhesive forces between the inner membranes 16 and the heart 11 as well as the negative pressure facilitate the heart 11 to expand in this manner. The inflatable membranes 16 also pull against the outer shell 14 and act to contract the outer shell 14. The outer shell 14 resists the contraction as a function of the elasticity of the outer shell's material and the spring constant of the flexible guides 22. Accordingly, the three dimensional stain forces applied to the heart during diastole are a combination of both the negative pressure created by the deflating inflatable membranes 16 and the resistance forces applied by the outer shell 14 and the flexible guides 22. Such three dimensional functionality can be assessed by a video camera of ultrasound probe directed by the guides or the tubing that delivers the pneumatic forces.

Since the cardiac assist device 10 is being applied to the heart 11, the heart 11 has either stopped pumping or is pumping in an ineffective manner. In order to have the heart 11 pump in an effective manner, forces have to be applied to the heart 11 during both the systole and diastole. The forces that need to be applied to the heart are referred to as the dynamic strain force requirements of the heart 11. These systolic and diastolic strain force requirements are three dimensional. The pneumatic forces delivered by the drive system combine with the inflatable membranes 16, and outer shell 14 strain characteristics which can be further modified by the flexible guides 22 to achieve the dynamic strain force requirements to properly pump the heart.

In the shown embodiment, four flexible guides 22 are shown. Such a number is exemplary, and it should be understood that one or more flexible guides can be used. The number of flexible guides 22, the diameter of the flexible guides 22, the material of the flexible guides 22, and the inherent shape of the flexible guides 22 all effect the resiliency of the flexible guides 22 and the manner in which the flexible guides 22 react when stressed.

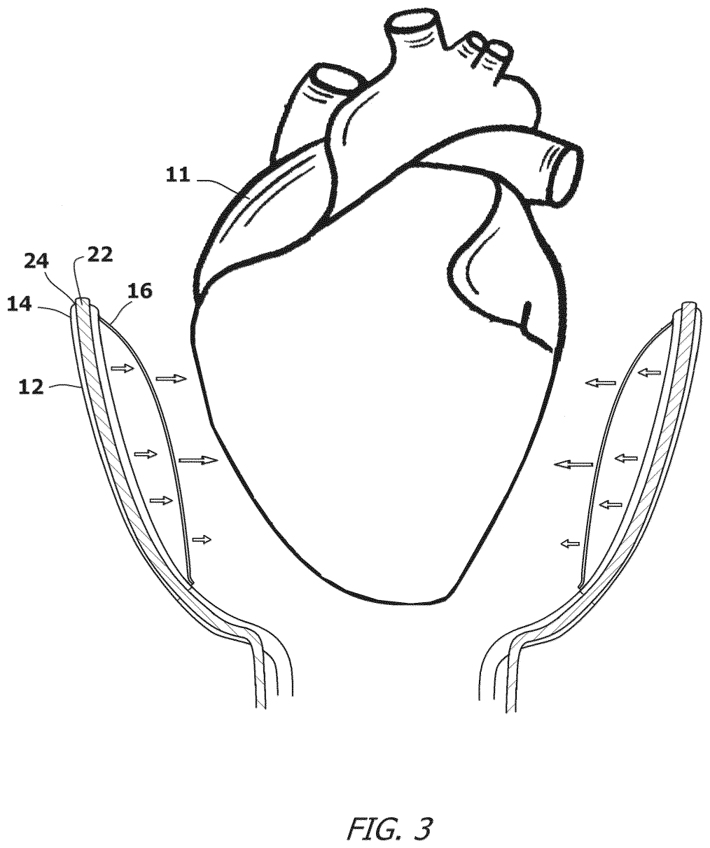

Referring to FIG. 3 in conjunction with FIG. 1 and FIG. 2, it will be understood that the flexible guides 22 pass through conduits 24 that are formed into the material of the outer shell 14. The outer shell 14 is flexible and is made from elastomeric material. Accordingly, the outer shell 14 has the ability to elastically expand, contract, elongate, shorten, and twist while being supported by the flexible guides 22. The resiliency of the flexible guides 22 and the elastomeric construction of the outer shell 14 enable the outer shell 14 to respond to physiological changes in the cardiovascular system that alter blood pressure and flow within the heart 11.

The inflatable membranes 16 are disposed in the interior of the outer shell 14. The inflatable membranes 16 are pneumatically or hydraulically inflated in a controlled manner that corresponds to the heart's cardiac cycle. The inflatable membranes 16 are also made from elastomeric material. The inflatable membranes 16 contact the epicardium of the heart 11. Due to the wet environment in vivo and the suction being applied to the cup structure 12, the elastomeric material of the inflatable membranes 16 adheres to the tissue of the heart 11 that is being contacted. As the inflatable membranes 16 are inflated by profiled drive system pressures, they 16 conform to the heart surface and apply either compressive or tensive forces to promote optimal cardiac pump function 11.

During the heart's cardiac cycle, a deficient or non-functioning heart will require assistance to pump efficiently. The assistance required by the heart can be quantified in terms of point displacement over time. That is, different points on the heart need assistance to move different distances at different times during the heart pumping cycle. Strain is the ratio of change in point positions over original point positions. As such, the assistance required by the heart can be quantified in terms of strain. The strains are dynamic and change over time with respect to the cardiac pumping cycle to for the heart to pump efficiently are herein referred to as the heart's strain force requirements. The strain force requirements of a heart are unique to that heart. If strain forces are applied to the heart that do not correspond to the needed strain force requirement, then the heart will not pump optimally which can mitigate the opportunity for recovery. This is also true if the strain forces being applied to the heart inhibits the optimal underlying pump function. These ideal strain requirements can be assessed by a small ultrasound probe or video camera inserted along the guides or within the drive tubing.

All forces applied to the heart 11 are applied through the application and operation of the cardiac assist device 10. The forces that the cardiac assist device 10 produce are dependent upon the strain characteristics of the inflatable membranes 16, the strain characteristics of the outer shell 14, the spring constants of the flexible guides 22, the number of flexible guides 22, the position of the flexible guides 22, and the inflation pressure profile used to selectively inflate/deflate the inflatable membranes 16. The strain characteristic of the inflatable membrane 16 and the outer shell 14 can be calculated using the techniques described in U.S. patent application Ser. No. 17/931,853 filed Sep. 13, 2022, and U.S. patent application Ser. No. 18/509,260 filed Nov. 14, 2023, the disclosures of which are herein incorporated by reference. The size and materials used for the flexible guides 22 are reflected in the spring constants of the flexible guides 22. Each spring constant is the ratio of the force acting on the flexible guide 22 in relation to the displacement of the flexible guide 22 caused by such forces.

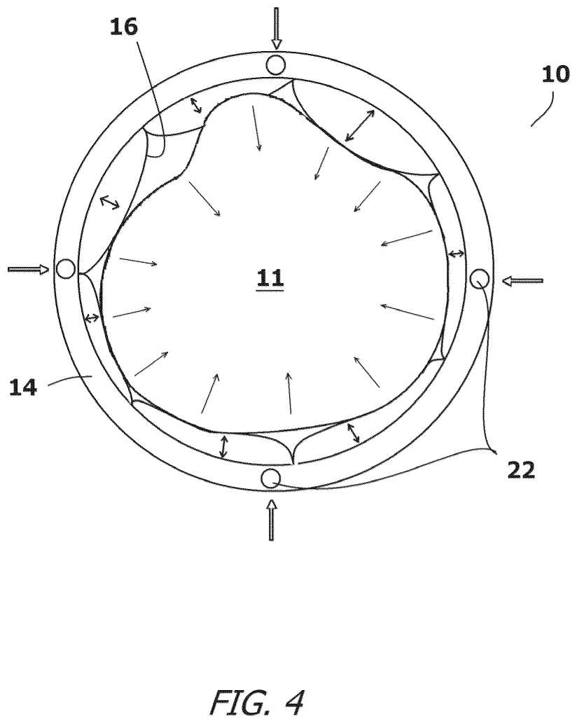

Referring to FIG. 4, it can be seen that when the heart 11 is emptying, the cardiac assist device 10 compresses the heart 11. The cardiac assist device 10 embodies overall dynamic strain characteristics that meet the strain force requirements of the heart 11 as it empties. To compress the heart 11, the inflatable membranes 16 inflate. The outer shell 14 prevents the inflatable membranes 16 from moving away from the heart 11 as they inflate and act upon the heart 11. The outer shell 14 is flexible and can move with the inflatable membranes 16 and the heart 11 as the heart 11 contracts, shortens, and twists. The outer shell 14 itself is reinforced by the flexible guides 22. The flexible guides 22 embody a spring constant. Accordingly, the flexible guides 22 can also move in response to the movements of the heart 11 and the inflatable membranes 16. The combined strain characteristics of the inflatable membranes 16 and the outer shell 14 added to the spring constants of the flexible guides 22 are matched as close as possible to produce the strain force requirements of the heart 11 when emptying.

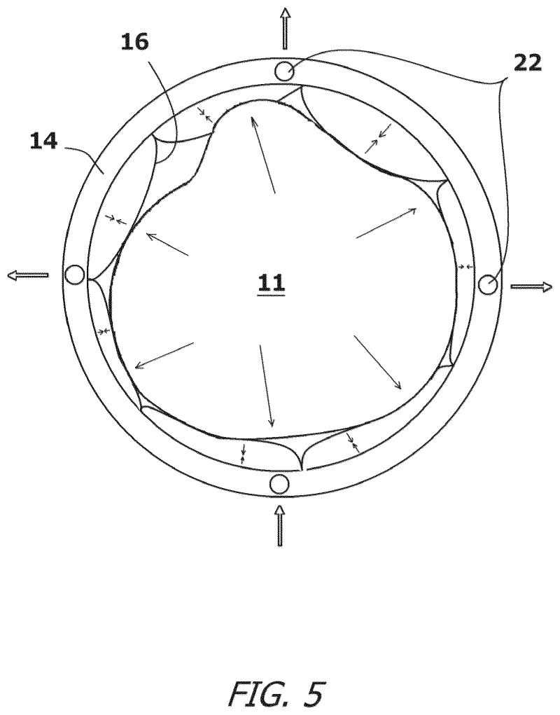

Referring to FIG. 5, it can be seen that when the heart 11 is filling, the cardiac assist device 10 helps the heart 11 expand. The cardiac assist device 10 embodies overall strain characteristics that meet the strain force requirements of the heart 11 as it fills. To help the heart 11 expand, the pressure in the inflatable membranes 16 is lowered. This creates low pressure between the heart 11 and the outer shell 14. The reinforcement provided by the flexible guides 22 prevent the outer shell 14 from collapsing toward the heart 11. Thus, the low pressure is maintained as the heart 11 fills. The combined strain characteristics of the inflatable membranes 16 and the outer shell 14 added to the spring constants of the flexible guides 22 are matched as close as possible to the strain assist profile required by the heart 11 when filling.

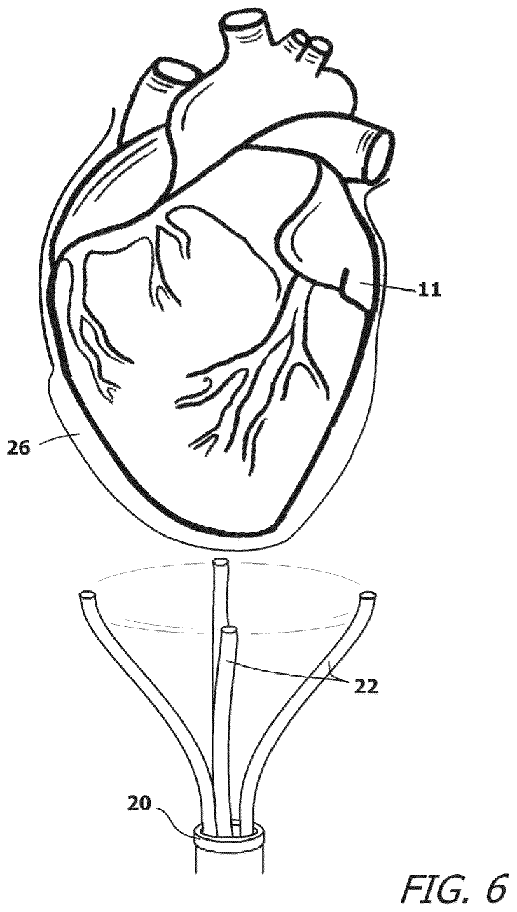

Referring to FIG. 6 in conjunction with FIG. 1, it will be understood that to utilize the present invention cardiac assist device 10, a prepared space 26 is created in the pericardial area to accommodate the cardiac assist device 10. The flexible guides 22 are then advanced into position within the prepared space 26. The processes needed to create the prepared space 26 and to position the flexible guides 22 into the prepared space 26 are described in U.S. patent application Ser. No. 18/447,786 filed Aug. 10, 2023, the disclosure of which is herein incorporated by reference.

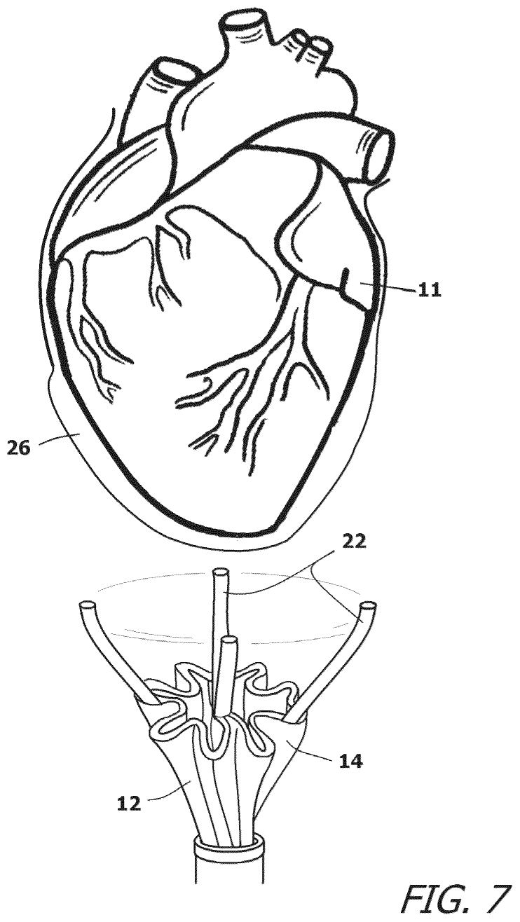

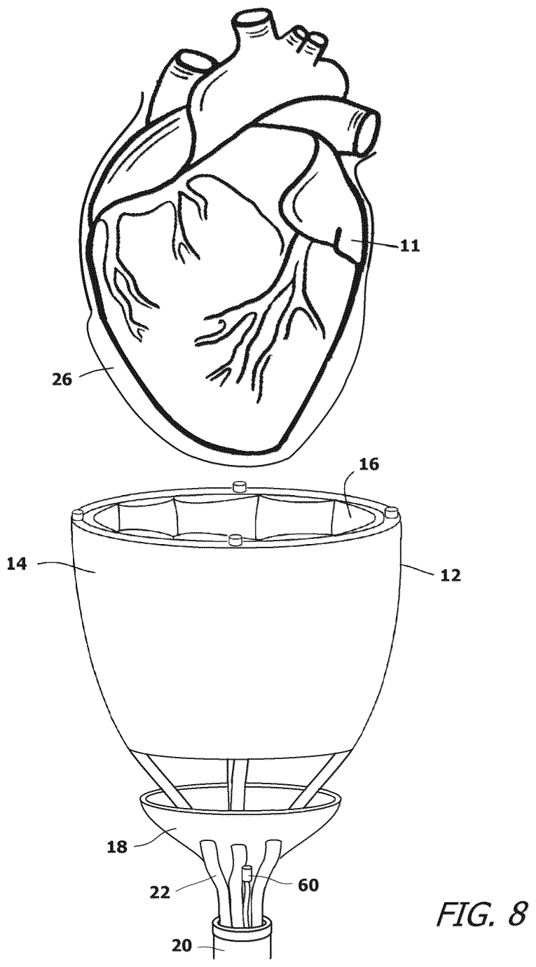

The flexible guides 22 are advanced into the pericardial area using an insertion tube 20 and minimally invasive surgical procedures. Referring to FIG. 7 and FIG. 8, it will be understood that the cup structure 12, which includes the outer shell 14 and the inflatable membranes 16, is advanced along the flexible guides 22 and into the prepared space 26, using the same insertion tube 20 as used with the flexible guides 22. The tubes 15 that provide suction and pneumatic pressure to the cup structure follow through the insertion tube 20 behind the cup structure 12. Once in position, the base 18 of the cup assembly 12 is also advanced along the flexible guides 22 and is connected to the bottom of the outer shell 14. Suction is supplied through the tube 15. The suction holds the base 18 against the cup structure 12 and helps advance the cup assembly 12 over the ventricles of the heart. The presence of the flexible guides 22 enables the cup structure 12 from collapsing under the forces created by the suction, therein enabling the cup structure 12 to expand and pass over the ventricles of the heart 11.

In FIG. 8, an optional view sensor 60 is shown. The view sensor can be advanced toward the heart 11 through the surgical insertion tube 20 or along one or more of the flexible guides 22. The view sensor 60 can be an ultrasonic sensor or a camera. The view sensor 60 can be used to determine the relative position between the cardiac assist device 10 and the heart 11 during installation. After installation, the view sensor 60 can be used to evaluate the functionality of the cardiac assist device 10 as it acts upon the heart 11.

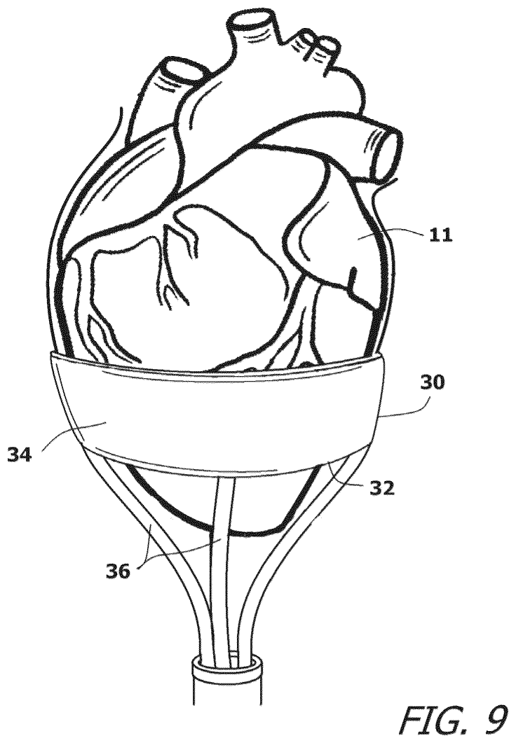

Referring to FIG. 9, an alternate embodiment of a cardiac assist device 30 is shown. In this embodiment, a cardiac assist device 30 is shown that provides an annular cuff 32 instead of a full cup. The annular cuff 32 has an outer shell 34 that encircles the heart 11. The outer shell 34 is structurally reinforced by the presence of flexible guides 36 that engage the outer shell 34.

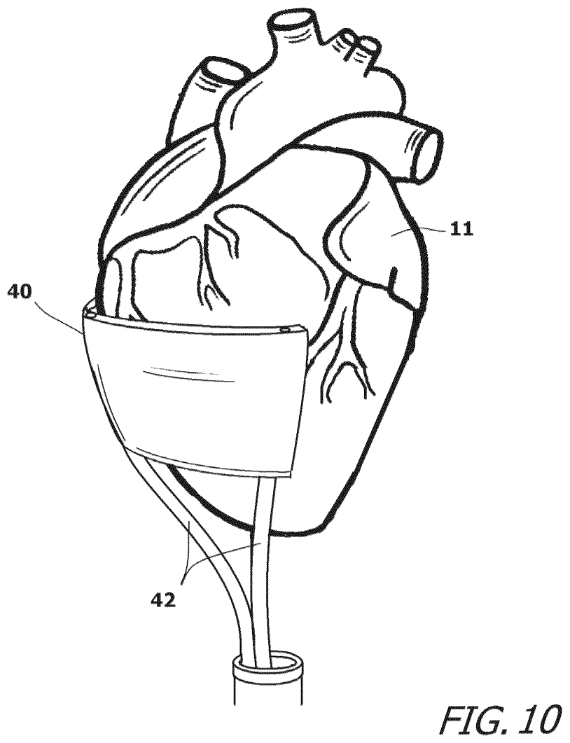

Referring to FIG. 10, a second alternate embodiment of a cardiac assist device 40 is shown that is designed to contact only one ventricular chamber of the heart 11. In this scenario, the placement of the cardiac assist device 40 is dependent upon flexible guides 42. Furthermore, the flexible guides 42 provide most all the forces needed to keep the cardiac assist device 40 in contact with the heart 11.

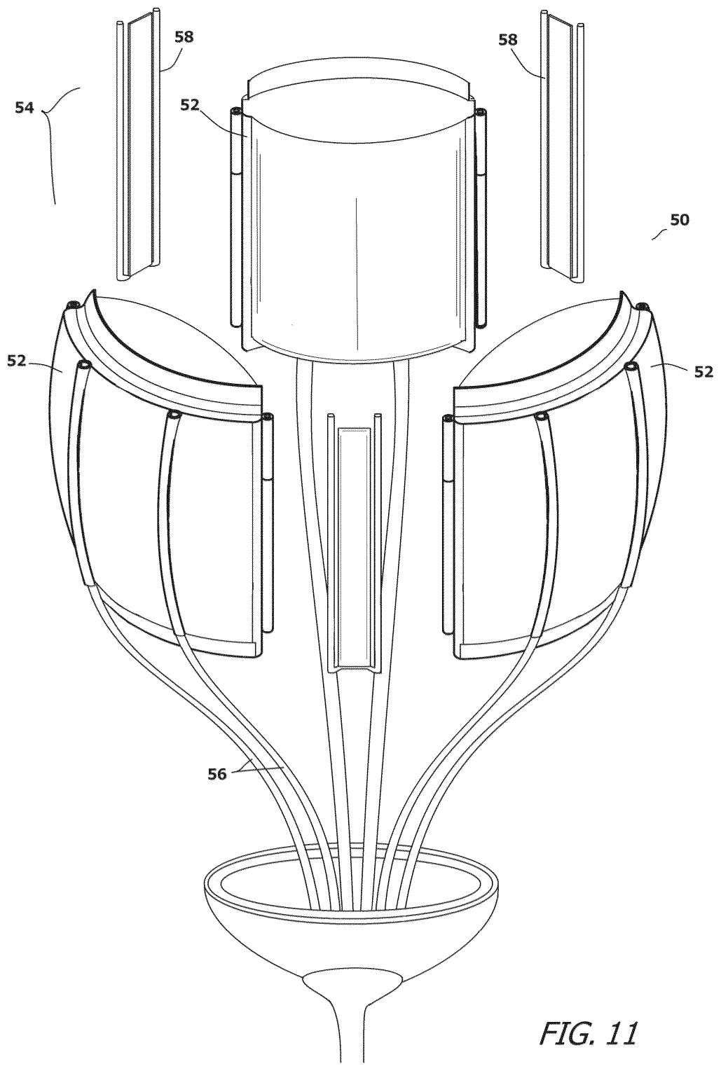

Referring to FIG. 11, a third embodiment of a cardiac assist device 50 is shown that is modular in design. A heart pump is formed in vivo by interconnecting modular segments 52 of a cup assembly 54. The structure for such a modular cup assembly is described in co-pending U.S. patent application Ser. No. 18/160,963, filed Jan. 27, 2023, the disclosure of which is herein incorporated by reference.

In the current adaptation, each of the modular segments 52 is supported by one or more flexible guides 56. The flexible guides 56 move and orient the modular segments 52 in vivo so that the modular segments 52 can be interconnected with connector panels 58. Once the modular segments 52 are interconnected into a full or partial cup structure, the flexible guides 56 provide the structural reinforcement needed for the cup assembly 54 to maintain position, resist collapse, and to apply the needed forces to the heart.

The use of modular segments 52 allows for the insertion of one module only in circumstances where the heart does not permit complete circumferential compression due to scarring or prior surgery, such as bypass grafts. An example of where a single module might be particularly useful is insertion on the back of the heart (posterior pericardial space) where the single module would act on the heart and have the advantage of the sternal bone providing a natural counter force. The sternal bone and associated anterior aspect of the heart is where scarring would be notable most prominent from any prior surgery and also where the most critical bypass graft (internal mammary artery) would lie. Modular concept would allow “building” around the heart with two or more modules to either partially or completed encompass ventricles.

It will be understood that the embodiments of the present invention that are illustrated and described are merely exemplary and that a person skilled in the art can make many variations to those embodiments. All such embodiments are intended to be included within the scope of the present invention as defined by the claims.

What is claimed is:

1. In a patient with a heart a systolic pumping capacity during systole and a diastolic pumping capacity during diastole, a method comprising:
advancing a plurality of flexible guides in vivo into positions proximate the heart;
providing a cardiac assist device;
advancing said cardiac assist device along said plurality of flexible guides into an operable position where said cardiac assist device can act upon the heart;
operating said cardiac assist device in vivo, wherein said cardiac assist device and said plurality of flexible guides in combination apply strain forces to the heart in systole and diastole that assist both said systolic pumping capacity and said diastolic pumping capacity.

2. The method according to claim 1, wherein providing a cardiac assist device includes providing a cardiac assist device with conduits through which said plurality of flexible guides pass.

3. The method according to claim 2, wherein said plurality of flexible guides are free to slide through said conduits in said construct.

4. The method according to claim 1, further including collapsing said cardiac assist device and advancing said cardiac assist device in vivo along said plurality of flexible guides in a collapsed configuration.

5. The method according to claim 4, wherein said plurality of flexible guides move said cardiac assist device from said collapsed configuration into said operable configuration in vivo proximate said heart.

6. The method according to claim 1, wherein said cardiac assist device has a cup structure with an outer shell that encircles at least one inflatable membrane, wherein said conduits are formed in said outer shell.

7. The method according to claim 6, wherein said cup structure has a base that attaches to said outer shell to form said cup structure, wherein said plurality of flexible guides pass through said base.

8. The method according to claim 6, wherein said outer shell, said at least one inflatable membrane, and said plurality of flexible guides all combine to supply controlled forces to the heart that are calculated to enhance a pumping efficiency associated with the heart.

9. The method according to claim 6, wherein said plurality of flexible guides prevent said outer shell from expanding beyond a first diameter and collapsing under a lesser second diameter when said cardiac assist device acts upon the heart.

10. The method according to claim 6, wherein said cardiac assist device has a collapsed configuration and an operable configuration, wherein said flexible guides maintain said construct in said operable configuration.

11. A method of advancing a cardiac assist device toward the heart, comprising:
advancing a plurality of flexible guides in vivo to positions about the heart;
advancing said cardiac assist device along said flexible guides in vivo to the heart; and
positioning said cardiac assist device into an operable position relative to the heart utilizing said plurality of flexible guides;
operating said cardiac assist device in vivo while said cardiac assist device is structurally reinforced by said flexible guides, wherein said plurality of flexible guides prevent said cardiac assist device from expanding beyond a first diameter and collapsing under a lesser second diameter when said cardiac assist device acts upon the heart.

12. The method according to claim 11, wherein said cardiac assist device has conduits through which said flexible guides pass.

13. The device according to claim 12, wherein said plurality of flexible guides are free to slide through said conduits in said construct.

14. The method according to claim 12, wherein said cardiac assist device has a cup structure with an outer shell that encircles at least one inflatable membrane, wherein said conduits are formed in said outer shell.

15. The method according to claim 14, wherein said cup structure has a base that attaches to said outer shell to form said cup structure, wherein said plurality of flexible guides pass through said base.

16. The method according to claim 11, further including providing a view sensor and advancing said view sensor along at least one of said plurality of flexible guides to detect a position of said cardiac assist device relative to the heart, wherein said view sensor is selected from a group comprising ultrasonic sensors and cameras.

17. The method according to claim 11, further including providing a view sensor and advancing said view sensor along at least one of said plurality of flexible guides to detect functionality of said cardiac assist device as said cardiac assist device acts upon the heart, wherein said view sensor is selected from a group comprising ultrasonic sensors and cameras.
